(19) United States
(12) Patent Application Publication
(10) Pub. No.: US 2026/0192013 A1
(43) Pub. Date: Jul. 9, 2026

(54) ADHESIVE TISSUE COVERING

(71) Applicant: Axogen Corporation, Alachua, FL (US)

(72) Inventors: Nesreen ALSMADI, Land O' Lakes, FL (US); Justin DEUERLING, Alachua, FL (US); Curt DEISTER, Gainesville, FL (US); Nikunj Kumar AGRAWAL, Tampa, FL (US); Benjamin Scott SPEARMAN, Alachua, FL (US)

(73) Assignee: Axogen Corporation, Alachua, FL (US)

(21) Appl. No.: 19/439,882

(22) Filed: Jan. 5, 2026

Related U.S. Application Data
(60) Provisional application No. 63/742,143, filed on Jan. 6, 2025.

(51) Int. Cl. A61L 15/58; A61F 13/0246

(52) U.S. Cl. CPC A61L 15/58; A61F 13/0253

(57) ABSTRACT

An adhesive tissue covering may include a base membrane and a biodegradable and biocompatible adhesive configured to bond to tissue. The adhesive may be located along one or more edges of the base membrane. In some aspects, an adhesive tissue strip may include a base membrane formed of at least one of a tissue-based material or a resorbable polymer, and a biodegradable and biocompatible adhesive configured to bond to tissue. The adhesive may be located on one side of the base membrane of the adhesive tissue strip.

### CROSS-REFERENCE TO RELATED APPLICATIONS

This application claims the benefit of priority to U.S. Provisional Patent Application No. 63/742,143, filed on Jan. 6, 2025, which is hereby incorporated by reference in its entirety.

### TECHNICAL FIELD

The present disclosure relates generally to the field of devices for the securing and fixing of tissue, and, more specifically, to an adhesive covering and an adhesive strip for the securing and fixing of tissue to other tissue and/or to other devices, and related methods of manufacture and use.

### BACKGROUND

Epineurial suture neurorrhaphy is widely acknowledged as the gold standard for peripheral nerve repair and reconstruction. This technique, however, requires significant expertise in microsurgical methods. Achieving proper micro suture placement and precise approximation of nerve ends are tasks that demand a high level of skill and are both time-intensive and technically challenging. Further, penetration of the suture needle beyond the outermost epineurial layer could result in damage to the critical fascicular structures of the nerve.

Given these inherent complexities, there is growing need for alternative tissue repair strategies, such as for nerves, that do not rely on sutures. Such innovations could significantly enhance the efficiency of tissue repair procedures, e.g., nerve repair procedures, by reducing the time required and simplifying the technical demands placed on surgeons.

### SUMMARY

In one aspect of the disclosure, an adhesive tissue covering may include a base membrane and a biodegradable and biocompatible adhesive configured to bond to tissue. The adhesive may be located along one or more edges of the base membrane.

In some aspects, an adhesive tissue strip may include a base membrane formed of at least one of a tissue-based material or a resorbable polymer, and a biodegradable and biocompatible adhesive configured to bond to tissue. The adhesive may be located on one side of the base membrane of the adhesive tissue strip.

Other objects, features and advantages of the present disclosure will become apparent from the following detailed description. It should be understood, however, that the detailed description and the examples, while indicating exemplary embodiments of the present disclosure, are given by way of illustration only, since various changes and modifications within the spirit and scope of the invention will become apparent to those skilled in the art from this detailed description.

In this disclosure, the singular forms “a,” “an,” and “the” include plural referents unless the context dictates otherwise. The term “exemplary” is used in the sense of “example” rather than “ideal.” The terms “comprises,” “comprising,” “includes,” “including,” or other variations thereof, are intended to cover a non-exclusive inclusion such that a composition, method, or process that comprises a list of elements or steps does not necessarily include only those elements or steps, but may include other elements or steps not expressly listed or inherent to such a composition, method, or process. The relative terms, such as “approximately” and “about,” are generally used to indicate a possible variation of ±10% of a stated or understood value unless indicated otherwise in the specification. In addition, the term “between” used in describing ranges of values is intended to include the minimum and maximum values described herein, unless noted otherwise. The use of the term “or” in the claims and specification is used to mean “and/or” unless explicitly indicated to refer to alternatives only or the alternatives are mutually exclusive, although the disclosure supports a definition that refers to only alternatives and “and/or.” As used herein “another” may mean at least a second or more.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of exemplary embodiments presented herein.

FIG. 1A is a schematic view of an adhesive covering, according to aspects of the disclosure.

FIG. 1B is a schematic view of an adhesive covering, according to aspects of the disclosure.

FIG. 2A is a schematic view of an adhesive covering, according to aspects of the disclosure.

FIG. 2B is a schematic view of an adhesive covering, according to aspects of the disclosure.

FIG. 3A is a schematic view of an adhesive covering, according to aspects of the disclosure.

FIG. 3B is a schematic view of an adhesive covering, according to aspects of the disclosure.

FIG. 3C is a schematic view of an adhesive covering, according to aspects of the disclosure.

FIG. 4 is a schematic cross-sectional view of an adhesive covering being applied to a nerve, according to aspects of the disclosure.

FIGS. 5A, 5B, and 5C are schematic views of example arrangements of adhesive on an adhesive covering, according to aspects of the disclosure.

FIG. 6 is a schematic view of an example arrangement of adhesive on an adhesive covering, according to aspects of the disclosure.

FIG. 7 is a schematic view of an exemplary adhesive covering with tabs, according to aspects of the disclosure.

FIG. 8 is an example arrangement of adhesive on an adhesive covering, according to aspects of the disclosure.

FIG. 9 is a schematic view of an adhesive covering with a liner, according to aspects of the disclosure.

FIG. 10A is a schematic side view of an adhesive layer on a base membrane, according to aspects of the disclosure.

FIG. 10B is a schematic side view of an adhesive layer on a mase membrane, and an optional time control layer on top of the adhesive layer, according to aspects of the disclosure.

FIG. 11A is a schematic view of an example of an adhesive covering with integrated adhesive strips, according to aspects of the disclosure.

FIG. 11B is a schematic view of the adhesive covering shown in FIG. 11A during use, according to aspects of the disclosure.

FIG. 11C is a schematic view of the adhesive covering shown in FIG. 11A during use, according to aspects of the disclosure.

FIG. 12A is a schematic view of an adhesive strip.

FIG. 12B is a schematic view of the adhesive strips of FIG. 12A in use.

FIG. 13A is an illustration of an example adhesive covering in the form of a wrap, according to aspects of the disclosure.

FIG. 13B is an illustration of an experimental use of the adhesive covering of FIG. 13A, according to aspects of the disclosure.

FIG. 13C is an illustration of experimental testing of the adhesive covering of FIG. 13A, according to aspects of the disclosure.

### DETAILED DESCRIPTION

Embodiments of the disclosure are drawn to an adhesive tissue covering, such as a nerve wrap. The adhesive tissue covering may be, e.g., in the form of a sheet, a pre-rolled sheet, or a tubular construct, and may be a nerve connector or a nerve protector, such as the Axoguard Nerve Connector®, the Axoguard Nerve Protector®, or the Axoguard HA+ Nerve Protector® by Axogen. Adhesive coverings of the disclosure may be used for fixation of tissue, and may be used to adhere to itself, to tissue onto which it is applied, or to a separate device. For example, transected nerve ends may be reconnected with the assistance of adhesive coverings of the embodiments. This includes the use of adhesive coverings in epineurial suture neurorrhaphy, as described above. One advantage of the present disclosure is enabling sutureless coaptation of a transected tissue, such as nerve tissue. In such a procedure, nerves may be joined together without use of traditional sutures, using adhesive coverings of the present disclosure. Or, if sutures are also used, fewer sutures may be needed as compared to the use of tissue coverings that are not adhesive. The embodiments of the present disclosure may be used to reduce reliance on traditional sutures for nerve repair and potentially enhance recovery outcomes. For example, having suture needles or barbs plunging into tissue may damage the tissue structures. In the case of nerves, suture needles or barbs may plunge into the body of the nerve and cause damage to nerve structures, which may inhibit nerve regeneration or repair and may further injure the nerve. Additionally, the advanced sutureless adhesive covering devices using biocompatible bioadhesives of the present disclosure may afford robust connection while being safe for the body and biodegradable without causing harm.

Aspects of the disclosure relate to an adhesive covering that allows for sutureless repair of injured tissue, including sutureless nerve repair of transected nerve ends or other nerve repair. The benefits of using adhesive coverings as described herein may include one or more of: improved patient outcomes for surgeons, including surgeons with minimal microsurgical training; saving operation room time, and thus money; improved or more consistent strength at the repair or coaptation site; and achieving a tissue connection device that does not risk further injury to the tissue from the use of suture needles. This may be particularly relevant for delicate nerve tissue, which is both a smaller and more sensitive type of tissue.

Embodiments of the present disclosure may include adhesive covering devices and methods for fabricating adhesive covering devices by applying a biodegradable bioadhesive to a base membrane of the adhesive covering. Thus, adhesive covering devices may include at least a base membrane and a biodegradable bioadhesive applied to the base membrane. In embodiments in which the adhesive coverings are intended for use with nerves, the bioadhesive may be configured to bond effectively with nerve epineurium. In such an example, when a side of a base membrane to which adhesive has been applied is contacted with a nerve, the bioadhesive may bond with the nerve epineurium, thus affixing the adhesive covering device to the nerve. This mode of attachment may be similar for use with other tissue types.

In some instances, the resulting adhesive covering devices may be described as sutureless nerve coaptation devices, although adhesive covering devices of the disclosure are not limited to use in coaptation procedures. For example, other nerve repair procedures, e.g., with a nerve that is injured but not fully transected, may be appropriate for use with the disclosed embodiments. Further, although nerve repair is used as an example throughout the description, such adhesive covering devices may be used in conjunction with other tissue types, as will be described further herein.

Embodiments may be drawn to base membranes in the form of nerve wraps having bioadhesive arranged thereon. Other embodiments may be drawn to a base membrane in the form of an adhesive strip having bioadhesive arranged thereon. These adhesive strips may be used alone or in conjunction with a nerve wrap, such as a nerve protector or a nerve connector, in order to keep the nerve wrap in place. In some embodiments of the present disclosure, the one or more adhesive strips may be integrated with the nerve wrap. Further, in one or more embodiments, the base membrane and/or adhesive may be biocompatible and resorbable. Additional aspects of the exemplary embodiments will be described herein in further detail.

According to some embodiments of the present disclosure, the base membrane, which may come in the form of a sheet (e.g., in the form of a strip or wrap), as a pre-rolled sheet, or as a tube (e.g., in the form of a connector), may be any suitable material for a tissue-repair procedure. In some examples, the base membrane may be extracellular matrix (ECM) material, such as small intestine submucosa (SIS), or other tissue-based materials. For example, the base membrane may be formed of amnion-based tissue (e.g., amniotic/chorionic membrane or amnion) and may be human or xenogenic, dermis, decellularized fascia, reconstituted denatured collagen, elastin, thrombin, fibronectin, starches, poly(amino acid), gelatin, alginate, pectin, fibrin, oxidized cellulose, chitin, chitosan, tropoelastin, hyaluronic acid, fibrin-based materials, collagen-based materials, hyaluronic acid-based materials, glycoprotein-based materials, cellulose-based materials, silks, or combinations thereof. In some aspects, the base membrane may be formed of a resorbable polymer membrane, such as one or more of homopolymers, copolymers, and/or polymeric blends including one or more of the following monomers: glycolide, lactide, caprolactone, dioxanone, trimethylene carbonate, monomers of cellulose derivatives, and monomers that polymerize to form polyesters. The polymers may include polydioxanone (PDS), polycaprolactone (PCL), polytrimethylene carbonate, polyglycolide (PGL), poly-3-hydroxybutyrate (PHB), poly(3-hydroxybutyrate-co-3-hydroxyvalerate) (PHBV), poly(propylene carbonate) (PPC), poly(butylene succinate) (PBS), poly(propylene fumarate) (PPF), polydroxyalkanoates, or one or more other suitable materials. In some examples, the base membrane may be configured to remodel into host tissue following implantation and may not leave permanent components, or significant amounts of permanent components, behind. The base membrane may provide circumferential coverage, or at least partial coverage, of a grafting site, coaptation site, injury site, or a repair site once applied, in an instance in which tubular-shaped tissues are the intended site of application (e.g., with nerves, blood vessels, etc.). The base membrane may protect the covered region of the tissue once applied, and, if used in the case of tissue transection, such as nerve transection, may protect the coaptation site from injured tissue bed.

According to some embodiments of the present disclosure, the adhesive may be biocompatible and biodegradable, i.e., the adhesive may completely or partially degrade over time and may not leave significant permanent material behind. The adhesive may be suitable for clinical adaptation. In some aspects, the adhesive may have a tunable degradation rate, i.e., the polymer composition may be modified to optimize the degradation rate in line with one or more of the tissue type to which the adhesive is intended to be applied to, or the intended use of the adhesive.

According to some aspects of the present disclosure, the adhesive may be a natural or a polymer adhesive. A polymer adhesive may be formed of one or more of, e.g., polylactic-co-glycolic acid (PLGA), polycaprolactone (PCL), Poly(lactic acid) (PLA), polyglycolic acid (PGA), poly(ε-caprolactone-co-lactide) (PCLA), polyhydroxyalkanoates (PHAs), or other suitable materials.

According to aspects of the disclosure, adhesive coverings may be formed by applying a biocompatible and biodegradable adhesive to a base membrane formed of natural or synthetic materials, as described above. The adhesive may be applied to the base membrane using various methods, such as painting, printing (e.g., 3D printing, inkjet printing, screen printing), rolling, spraying, solvent casting, or any other methods of applying the adhesive. In some aspects, the adhesive may be applied to a base membrane using stencils. Such application methods may promote consistent deposition of adhesive material and scalable manufacturing. This adhesive, once applied, may be used to secure the base membrane to tissue, such as a nerve, or to itself.

The adhesive may be applied to different regions of the base membrane and may be applied in different shapes and sizes. In some examples, the adhesive may be applied as a linear strip, or as a series of linear strips, e.g., to one or more side or edge regions of the base membrane. In other aspects, adhesive may be applied in a series of discrete shapes or as a regular or irregular pattern of discrete shapes, e.g., as dots, drops, triangles, quadrilaterals, lines, star-with-dot motifs, zigzags, etc. In some aspects, adhesive may be applied in serpentine patterns, as a continuous serpentine line or as a series of discrete lines, or as any other suitable shapes or arrangements. In some aspects, the size, shape, arrangement, or distribution of adhesive may be determined, at least in part, on the type of tissue to which the adhesive covering is intended to be applied, or the particular use case for the adhesive covering. The primary function of the adhesive portion may be to secure the base membrane (e.g., a wrap, a protector, or a connector) in place, promoting stable positioning and alignment.

Still further, in some aspects, adhesive may only be applied to one side of a base membrane. For example, the base membrane may include a first surface and a second surface, and the adhesive may only be applied to one of the first surface and the second surface, but not both. In such embodiments, the single-sided nature of the adhesive may inhibit unnecessary sticking to adjacent tissues or structures once the adhesive covering is applied to a tissue such as a nerve. Alternatively, in other aspects, adhesive may be applied to one or more portions on both sides of the base membrane. For example, adhesive may be applied to portions of the base membrane configured to overlap with one another portion of the base membrane once the base membrane is applied to the tissue.

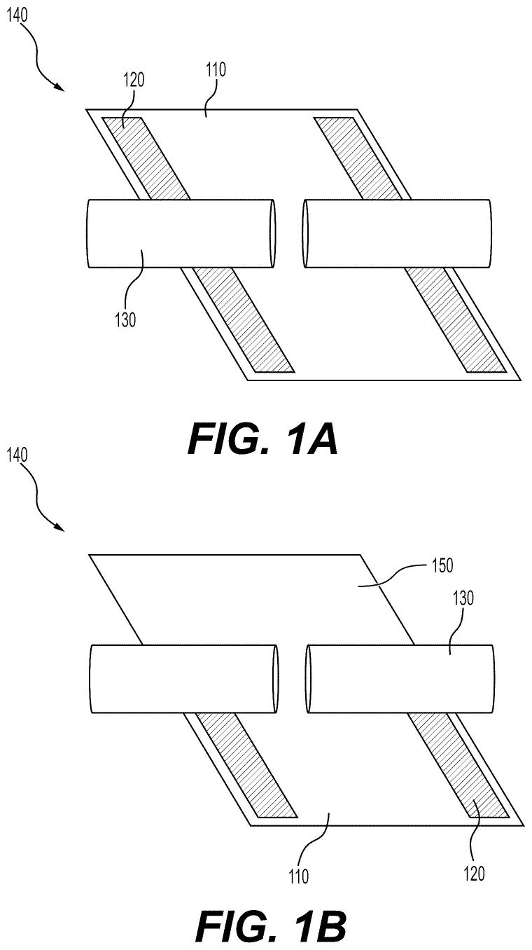

In some examples, the adhesive may be arranged on the base membrane in various configurations to optimize the coaptation strength of, e.g., transected nerve ends, as shown in FIGS. 1A, 1B, 2A, 2B, 3A and 3B. The arrangement, shape, size, or locations of the adhesive on the base membrane may be selected based on the amount of adhesion strength needed for the intended use case. For example, the adhesive may be applied so as to provide robust strength during nerve coaptation or nerve repair. The customization may allow adhesive coverings to be effective in securing the covering in place while being gentle enough to inhibit risk of damage to the tissue, e.g., in the case of delicate nerve tissue, to which it is applied. For example, the retention strength of the adhesive may be similar to that of 9-0 gauge sutures. In some aspects, as described further below, the adhesive may be arranged on the base membrane so as to not interfere with the site of injury on the tissue. In the case of a transected nerve, the adhesive may be positioned on the base membrane so as to not interfere with the face of the transected nerve stump.

In all of the embodiments described herein, the adhesive may have a clear color, a color that is similar to the base membrane or base layer, or a color that is different from the base membrane or base layer. In some aspects, an adhesive may have a color that is different from the base membrane or base layer so as to allow the user to see where the adhesive is on the device, and where to apply the device to the tissue. The different color may enhance visibility and help a user to determine the location of the adhesive prior to application of the device to tissue during use. The difference in color may also allow a user to identify which side of the adhesive covering is the sticky side. In some aspects, the color may change to indicate to a user that the adhesive has set, and thus the user should no longer try to reposition the adhesive covering. For example, in the case of nerves, the change in color once the adhesive has set may prevent a user from trying to reposition the adhesive covering, which may otherwise damage the underlying tissue, such as the delicate epineurium layer in the case of nerve tissue.

FIG. 1A depicts an example of the adhesive 120 applied along an edge of the base membrane 110. In the example of FIG. 1A, the edge to which the adhesive 120 is applied is configured to wrap around or otherwise overlay the tissue to which the adhesive covering 140 is applied. In the case of the tissue depicted in FIG. 1A, the adhesive edge of the adhesive covering 140 may wrap around an outer circumference of nerve 130. Adhesive 120 may be applied along the entire edge of the base membrane 110, or to less than the entire edge. Similarly, the adhesive may extend up to the extreme edge of the base membrane 110, or may be set back from the extreme edge of the base membrane 110. As shown in FIG. 1A, the adhesive 120 may be applied to one edge, or to more than one edge of base membrane 110, e.g., to opposite edges of base membrane 110, as shown in FIGS. 1A and 1B.

FIG. 1A depicts an example method use of adhesive covering 140. In this example, transected tissue, e.g., nerve tissue, may be positioned with a slight gap between the two transected portions. The portions of the adhesive covering 140 to which the adhesive 120 is applied (in this case, the two opposing edges) may be oriented so that portions of the transected tissue set back from the transected ends are overlaid with the adhesive 120 on the base membrane 110. The adhesive 120 along one edge of the base membrane 110 may be applied to one portion of nerve 130 at a region proximal to the distal-most transected end of the first portion, and adhesive 120 on the opposite side of the base membrane 110 may be applied to a second portion of nerve 130 at a region proximal to the distal-most transected end of the second portion. The adhesive covering 140, in this case, a wrap in the form of a sheet, may be wrapped around both portions of nerve 130, so that the entire circumference of the nerve ends are covered, or so that at least a portion of the nerve ends are covered. The adhesive covering 140 may overlap on itself or may not overlap on itself and may only overlay the portions of nerve 130. If the adhesive covering 140 overlaps with itself, the adhesive 120 may function so as to attach adhesive covering 140 to the underlying portions of nerve 130 where it contacts nerve 130, as well as function so as to adhere covering 120 to itself to hold adhesive covering 140 in place once it has been wrapped. Once the adhesive covering 140 is applied, it may maintain the two portions of the transected nerve 130 in position relative to one another and hold them together. The portion of adhesive covering 140 that encloses or overlays the gap between the two transected nerve portions may protect, guide, or enclose outgrowing axons. This may inhibit outgrowing axons from growing somewhere other than the other transected nerve end, and may inhibit exposure of the injured nerve ends or outgrowing axons to other surrounding structures, or anything in the surrounding environment.

By including adhesive 120 along opposing edge portions of base membrane 110, and not in a central region of base membrane 110, where the gap between the portions of nerve 130 are oriented during use, the adhesive 120 may not interfere with the coaptation site. This may be desirable, as adhesive 120 may interfere with outgrowing axons. Further, if the sidewalls of the wrapped adhesive covering 140 were to contact each other in the area that overlies the gap between the two nerve portions after being applied to nerve portions, and if the sidewalls were covered with adhesive in that region, then the side walls may stick together and thus cut off communication between the two transected nerve ends, which may inhibit the repair of the nerve portions and prevent axons from one transected nerve end from reaching the other transected nerve end. In some instances, this could further lead to the formation of neuromas. In other aspects, such as use with injured but not transected tissues, not having adhesive in a region designed to overlay a site of injury or repair may similarly inhibit irritation or interference with healing of the underlying injured region. In still other aspects, however, it may be acceptable for adhesive 120 to be located in a central region of base membrane 110, and not just edge regions of base membrane 110.

Accordingly, adhesive coverings may be divided into adhesive sites and coaptation or injury sites. The adhesive sites may include the biocompatible adhesive, and the coaptation or injury sites may have no adhesive and may be the target zones for where the nerve ends or tissue ends, or otherwise injured or repaired tissue, may be oriented during use.

In some examples, on the side of the adhesive covering without adhesive, a lubricating layer may be applied to further prevent unwanted sticking of the adhesive covering to other surfaces near the site of tissue repair, e.g., nerve coaptation. In some examples, the lubricating layer may include hyaluronate or alginate. Suitable lubricating layers may be described in U. S Patent Application Publication No. 2021/0046221, filed Aug. 13, 2020, which is incorporated by reference in its entirety herein. Such lubricating layers may be included on the non-adhesive sides of any of the adhesive coverings described in this disclosure and depicted in FIGS. 1A through 11C.

Although use with transected tissue, and specifically transected nerve portions, is described above, tissue need not be fully transected in order to be suitable for use with embodiments of the disclosure. For example, partially transected tissues, or damaged or sensitive tissues, e.g., nerve tissues, may be used with embodiments of the disclosure.

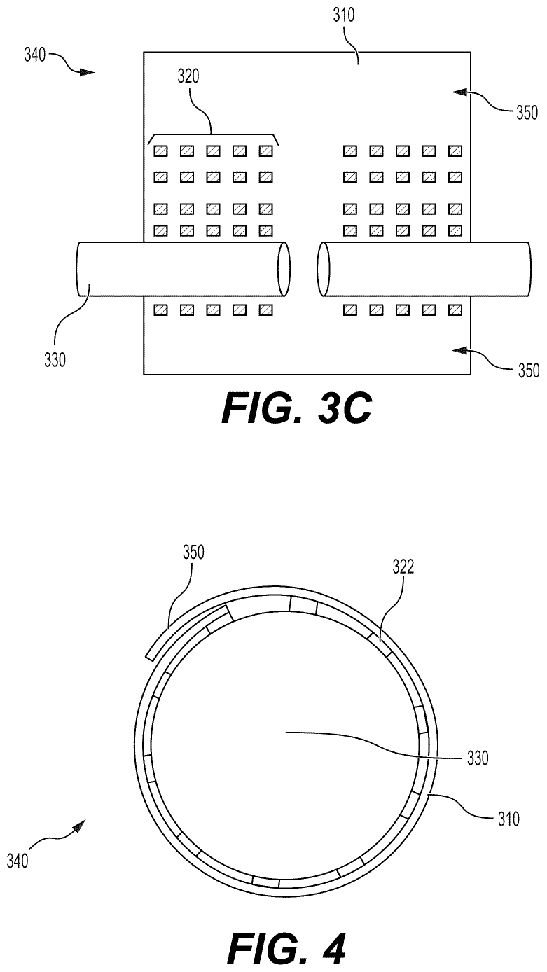

FIG. 1B depicts another example of an adhesive covering 140 in the form of a wrap with the adhesive 120 applied along an edge, and specifically, along two opposing edges, of the base membrane 110. In FIG. 1B, adhesive 120 is applied to less than all of the edge of the base membrane 110. In some aspects, adhesive 120 may be applied to about ¾ of the edge of base membrane 110, about ½ of the edge of base membrane 110, about ¼ of the edge of base membrane 110, or less than half of the edge of base membrane 110. Accordingly, an adhesive-free zone 150 may be formed on a portion of base membrane 110. Adhesive-free zone 150 may allow for a user to more easily handle adhesive covering 140, e.g., with their fingers, forceps, or other surgical instruments, e.g., when applying adhesive covering 140 to tissue without smearing or otherwise disturbing adhesive 120 or causing adhesive 120 to become less sticky. Further, adhesive-free zone 150 may allow adhesive covering 140 to be wrapped around tissue without different portions of base membrane 110 being adhered to itself, as is described in further detail below in reference to FIG. 4.

In this embodiment, the portion of adhesive covering 140 to which adhesive 120 is applied may be wrapped around the transected portions of nerve 130, or otherwise secured to the relevant portion of underlying tissue, and the portion of adhesive covering 140 that does not include adhesive 120 may be wrapped on top of underlying portions of adhesive covering 140. Accordingly, when in use, the adhesive sites would attach to the nerve tissue 130. Otherwise, the wrapping and attachment of the adhesive covering 140 of FIG. 1B would be similar to what is described in reference to FIG. 1A, and may be used for similar use cases. Although FIGS. 1A and 1B depict adhesive 120 extending along edges of base membrane 110 that are generally perpendicular to a longitudinal axis of the tissue to which adhesive covering 140 is being applied, in other aspects, adhesive 120 may be included along one or more edges of base membrane 110 that extend generally parallel to a longitudinal axis of the tissue to which adhesive covering 140 is being applied, or in any other relative orientation.

As described above, adhesive 120 of FIGS. 1A and 1B may be applied to base membrane 110 using one or more various methods, such as painting, printing (e.g., 3D printing, inkjet printing, screen printing), rolling, spraying, solvent casting, or any other methods of applying the adhesive. In some aspects, the adhesive may be applied to a base membrane using stencils to create reproducible patterns of adhesive on the base membrane. Such application methods may promote consistent deposition of adhesive material and scalable manufacturing.

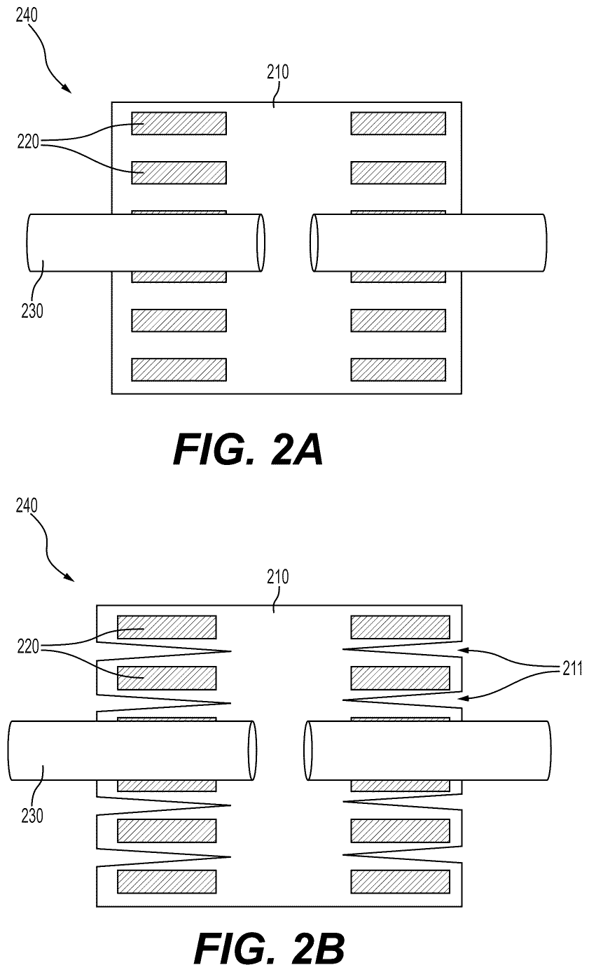

FIG. 2A depicts an example of an adhesive covering 240 to which adhesive 220 is applied in discrete strips along one or more edges of base membrane 210. In the embodiment of FIG. 2A, the discrete strips of adhesive 220 are oriented generally perpendicular to the edge on which they are applied, however, other orientations may be suitable. Including discrete strips or discrete islands of adhesive 220 may allow for more movement, flexibility, shrinking, or inflammation of the underlying tissue once the adhesive covering 240 is implanted on the tissue. For example, if the tissue is a nerve 230, and the nerve is in a part of the body that rotates, bends, or otherwise stretches or contracts, then discrete portions of adhesive 220 may allow for more freedom of movement of the underlying tissue while still maintaining coverage of the tissue and/or keeping the portions of tissue in place relative to one another. As another example, injured tissue may experience swelling after the adhesive covering 240 has been applied, and discrete portions of adhesive 220 may allow for more expansion or stretch of the base membrane 210 without compressing the underlying nerve. Still further, the injured tissue to which adhesive covering 240 has been applied may be swollen when adhesive covering 240 is applied to the tissue, and the tissue may shrink after the application process. Again, having discrete portions of adhesive 220 instead of a continuous application of adhesive 220 may allow the adhesive covering 240 to contract more easily without pulling on the underlying tissue, e.g., the delicate nerve epineurium. For example, the base membrane may be able to pucker, bunch, cinch, or otherwise flex to allow for the adhesive portions to move closer to one another as the underlying tissue shrinks, in turn allowing the adhesive covering 240 to remain attached to the tissue as the tissue retracts away from it. In some aspects, after application of the adhesive covering 240, the underlying tissue may swell and then subsequently contract, all while the adhesive covering 240 is still applied.

FIG. 2B depicts an example of the adhesive covering 240 with slits 211 in the base membrane 210. The slits 211 may be included to further allow for expansion, contraction, flexing, rotating, or other movement of the underlying tissue, e.g., nerve 230, after application of adhesive covering 240, similar as to described above in reference to FIG. 2A. For example, swelling, shrinking, flexion may occur between the adhesive sites in the location of the slits. The base membrane on either side of a given slit may more easily be able to move towards or away from each other, or to splay out or bend, even if there is less give or stretchiness to the material from which the base membrane 210 is formed. Adhesive 220 may be applied on either side of slits 211. In the example shown in FIG. 2B, the adhesive 220 may be applied to the base membrane 210 in longitudinal strips, generally perpendicular to an edge of base membrane 210 along which the adhesive 220 is applied, on either side of the slits 211. Alternatively, the adhesive 220 may be applied in any other suitable discrete portions on either side of slits 211. Further, although the slits are shown as creating narrow, triangular gaps in the base membrane 210, in some aspects, gaps of different shapes or sizes may be created. In other aspects, the slits may not create gaps and may be formed as cuts in the base membrane 210.

Although FIGS. 2A and 2B depict adhesive 220 extending along an entirety of one or more edges of base membrane 210, in some aspects, adhesive 220 may extend along less than the entirety of one or more edges, such as shown in FIG. 1B. Accordingly, in some aspects, the adhesive coverings 240 of FIGS. 2A and 2B may also include adhesive-free zones, which may facilitate handling or application as described above.

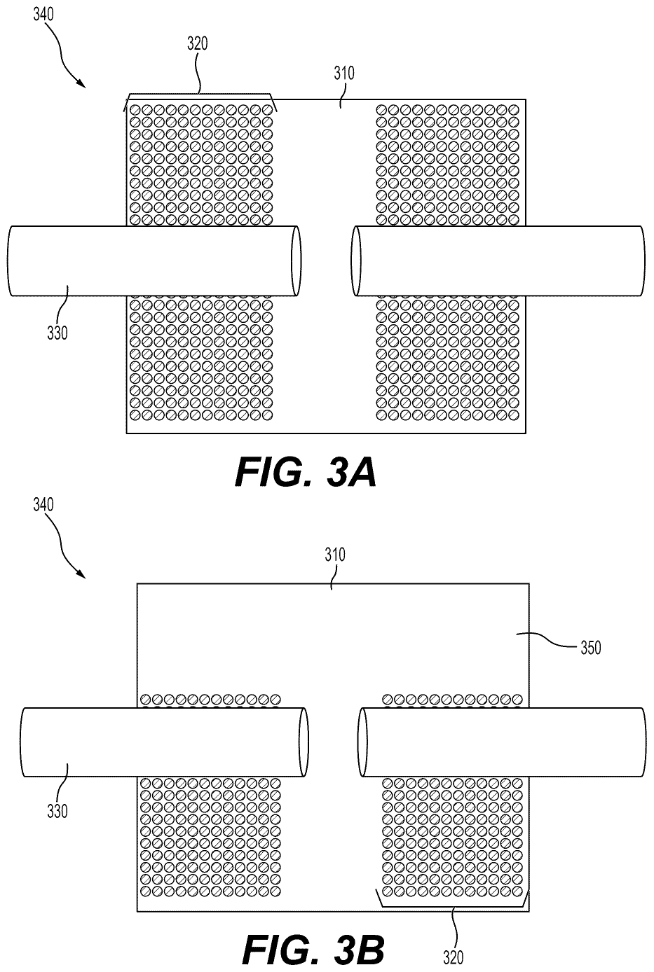

Although the exemplary embodiments of FIGS. 1A, 1B, 2A, and 2B depict linear portions of adhesive 220 along one or more edges of base membrane 110, as referenced above, adhesive 220 may be applied in a series of linear portions, in one or more non-linear portions (e.g., serpentine, irregular, or differently shaped portions) or in other suitable configurations. In other aspects, adhesive 220 may be applied in a series of discrete islands, as will be described further in reference to FIGS. 3A-7.

Although FIGS. 2A and 2B depict adhesive 220 extending along edges of base membrane 210 that are generally perpendicular to a longitudinal axis of the tissue to which adhesive covering 240 is being applied, in other aspects, adhesive 220 may be included along one or more edges of base membrane 210 that extend generally parallel to a longitudinal axis of the tissue to which adhesive covering 240 is being applied, or in any other relative orientation.

As described above, adhesive 220 of FIGS. 2A and 2B may be applied to base membrane 210 using one or more various methods, such as painting, printing (e.g., 3D printing, inkjet printing, screen printing), rolling, spraying, solvent casting, or any other methods of applying the adhesive. In some aspects, the adhesive may be applied to a base membrane using stencils to form reproducible patterns of adhesive on the base membrane. Such application methods may promote consistent deposition of adhesive material and scalable manufacturing.

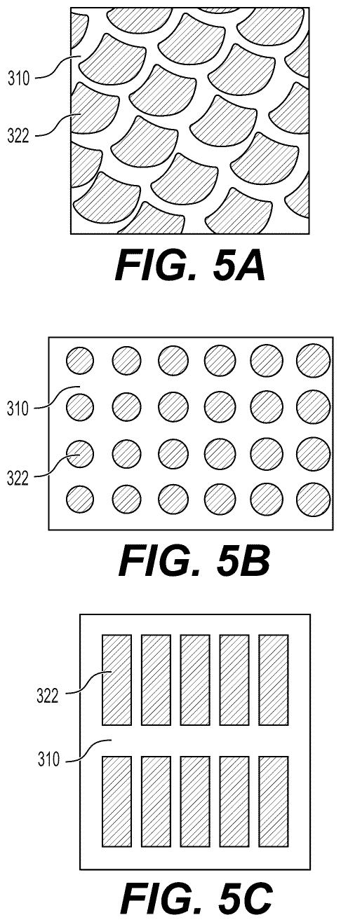

FIGS. 3A-3C depict examples of an adhesive nerve covering 340 on which the adhesive 320 is applied to the base membrane 310 in discrete islands. In some examples, the discrete islands may be irregular droplets, circles, rectangles, triangles, star-with-dot motifs, serpentine shapes, linear shapes, or any other suitable shape. The discrete islands of the adhesive 320 may be deposited randomly or in a regular pattern, such as rows and columns, offset rows, or any other suitable arrangement. For example, FIG. 5A depicts fan-shaped discrete islands 322 of adhesive arranged in offset rows, FIG. 5B depicts circular discrete islands 322 arranged in rows, and FIG. 5C depicts rectangular discrete islands 322 arranged in rows. The regular or irregular patterns of discrete islands 322 may be arranged on any suitable portion of base membrane 310. In FIG. 3A, the adhesive 320 is applied to the base membrane 310 along the edge of the base membrane 310 configured to wrap around or otherwise attach to the underlying tissue to adhere the adhesive covering 340 to the tissue.

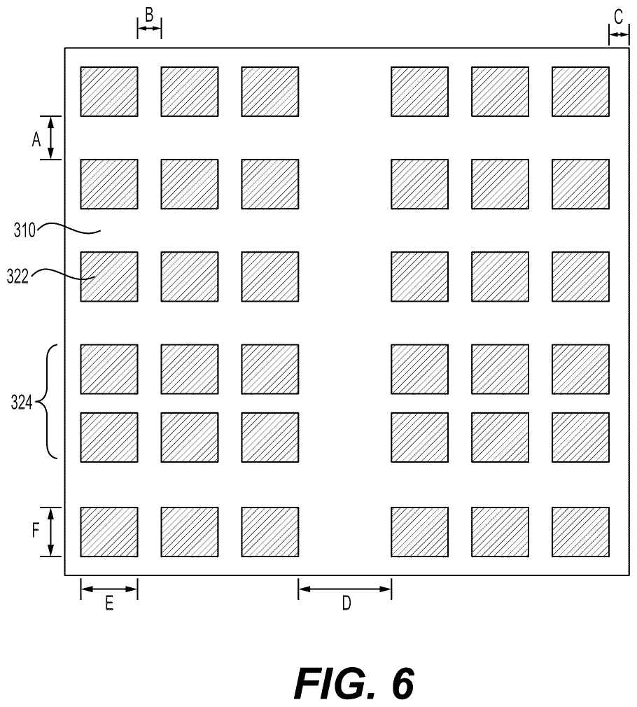

As described in reference to FIG. 1A, adhesive 320 may be applied along the entire edge of the base membrane 310, or to less than the entire edge. Similarly, the adhesive 320 may extend up to the extreme edge of the base membrane 310, or may be set back from the extreme edge of the base membrane 310, as will be discussed in reference to FIG. 6. As shown in FIG. 1A, the adhesive 320 may be applied to more than one edge of base membrane 310, e.g., to opposing edges of base membrane 310, or may be applied to only one edge of base membrane 310. In some aspects, adhesive 320 may not be applied to a central portion intended to overlay a gap between two transected tissue, e.g., nerve, ends 330, or other areas of tissue repair or injury. Adhesive covering 340 may be applied to the nerve ends 330, or any other suitable tissue whether transected or not, as described in reference to FIG. 1A, above.

FIG. 3B depicts an embodiment similar to FIG. 1B, in which adhesive 320 is applied to less than an entire edge of the base membrane 310 of adhesive covering 340. In the embodiment of FIG. 3B, as in FIG. 3A, the adhesive 320 is applied in a pattern of discrete islands. In some aspects, adhesive 320 may be applied to about ¾ of the edge of base membrane 110, about ½ of the edge of base membrane 110, about ¼ of the edge of base membrane 110, or less than half of the edge of base membrane 110. Accordingly, an adhesive-free zone 350 may be formed on a portion of base membrane 310. Adhesive-free zone 350 may allow for a user to more easily handle adhesive covering 340, e.g., with their fingers, forceps, or other surgical instruments, e.g., when applying adhesive covering 340 to tissue without smearing or otherwise disturbing adhesive 320 or causing adhesive 320 to become less sticky. Further, adhesive-free zone 350 may allow adhesive covering 340 to be wrapped around tissue without different portions of base membrane 310 being adhered to itself, as is described in further detail below in reference to FIG. 4.

In this embodiment, the portion of adhesive covering 340 to which adhesive 320 is applied may be wrapped around the transected portions of nerve 330, and the portion of adhesive covering 340 that does not include adhesive 320 may be wrapped on top of underlying portions of adhesive covering 340, as described in reference to FIG. 1B. Accordingly, when in use, the adhesive sites would attach to the nerve portions 330 in the example of FIG. 3B. Otherwise, the wrapping and attachment of the adhesive covering 340 of FIG. 3B would be similar to what is described in reference to FIG. 1A, and may be used for similar use cases. Although FIGS. 3A and 3B depict adhesive 320 extending along edges of base membrane 310 that are generally perpendicular to a longitudinal axis of the tissue to which adhesive covering 340 is being applied, in other aspects, adhesive 320 may be included along one or more edges of base membrane 310 that extend generally parallel to a longitudinal axis of the tissue to which adhesive covering 340 is being applied, or in any other relative orientation.

FIG. 3C depicts another example embodiment in which adhesive 320 is applied to base membrane 310 such that adhesive-free zones 350 are formed on both sides of base membrane 310. In such embodiments, adhesive-free zones 350 may be the same size or may be different sizes relative to one another. Adhesive covering 340 may otherwise be similar to adhesive coverings 3A and 3B.

As described above, adhesive covering 340 may be wrapped around tissue, such as a nerve 330 or other tubular-shaped tissue. FIG. 4 is a schematic, cross-sectional view of a nerve 330 wrapped with adhesive covering 340. Discrete islands 322 of adhesive may contact the underlying tissue and adhere base membrane 310 to nerve 330. Depending on the height of discrete islands 322 of adhesive, a slight gap may be formed between base membrane 310 and the underlying tissue. Even if no visible gap is formed, the base membrane 310 between discrete islands 322 of adhesive may not be directly adhered to the underlying tissue. In some aspects, as shown in FIG. 4, adhesive-free zone 350 may be overlapped with another portion, e.g., the opposite edge, of adhesive covering 340. By overlapping adhesive-free zone 350 in such a manner, base membrane 310 of adhesive covering 340 may not be adhered to itself. Instead, base membrane 310 may be free where it wraps around itself. This may allow the underlying tissue to swell after application of adhesive covering 340 by not setting or restricting the circumference of adhesive covering 340. Allowing for swelling of the underlying tissue may inhibit compression of the tissue and thus inhibit potentially worse surgical outcomes as a consequence.

In some aspects, one or more of the density, size, thickness, shape, or other properties of the discrete islands may be adjusted to balance retention strength, e.g., coaptation strength, during use, with ease of application of the adhesive covering to tissue. In some aspects, the density, size, thickness, shape, or other properties of the discrete islands may be adjusted so as to meet a minimum threshold for retention strength (e.g., coaptation strength) while allowing for more room between the discrete islands of adhesive to permit swelling and contraction of the underlying tissue. For example, the discrete islands of adhesive 320 may be spaced closer or further apart from one another, as will be described further below in relation to FIG. 6. As one example, the use of rectangular discrete islands may optimize the surface area covered by adhesive 320 to promote retention strength, while still allowing for space between adjacent rectangular discrete islands.

In some aspects, the relative spacing or arrangement of discrete islands of adhesive may allow for nutrient and oxygen diffusion to cells, which may facilitate and accelerate cell infiltration and tissue ingrowth or remodeling. Additionally or alternatively, the relative spacing or arrangement may allow for regulation of cellular waste and degradation byproducts of adhesive 320 over time, which may facilitate a healthy microenvironment and removal of any unwanted byproducts. For example, arranging the discrete islands in rows or columns may result in a pattern of interconnected channels between discrete islands 322 of adhesive, which may run in longitudinal and latitudinal directions. FIG. 6 shows such an exemplary arrangement.

FIG. 6 depicts an exemplary base membrane 310 with rows and columns of discrete islands 322 of adhesive included on a surface thereof. In FIG. 6, columns of discrete islands 322 of adhesive are generally spaced apart from each other by a distance B, and rows of discrete islands 322 of adhesive are generally spaced apart from each other by a distance A. In one example, distance A may be approximately 0.5 mm to approximately 2 mm, e.g., approximately 0.5 to approximately 1 mm, approximately 1 mm to approximately 2 mm, or approximately 0.75 mm to approximately 1.5 mm. In general, distance A may be sufficiently large to facilitate optional use of forceps or other surgical tools by a clinician to create space between an underlying tissue and the adhesive covering device to allow unrestricted swelling of the tissue to which the adhesive covering is applied. Minimum distance A may be at least partially governed by practical limitations for how closely discrete islands 322 of adhesive may be applied without overlapping with neighboring discrete islands 322. Increasing distance A too much may reduce the overall amount of adhesive on base membrane 310, and thus the retention strength of the adhesive tissue covering during use, which may negatively impact the utility of the adhesive covering.

In some aspects, two or more rows of discrete islands 322 of adhesive may be spaced apart from one another an amount that is less than distance A, for example, at landing zone 324. Landing zone 324 may function as an initial point of contact with a tissue to which base membrane 310 will be attached. For example, in the case of a small tissue, such as a nerve, the clustering of discrete islands 322 of adhesive in landing zone 324 may make it easier for a user to position the nerve on the base membrane 310 in such a way that the nerve contacts with and sticks to discrete islands 322 of adhesive to adhere the base membrane 310 to the nerve. While spacing A may be well suited to allow for tissue swelling and contraction, the flow of nutrients, the flow of waste, and manipulation of base membrane 310 with forceps, it may be more difficult to position the nerve relative to the adhesive covering so that it adheres to base membrane 310 if the rows of discrete islands 322 of adhesive are spaced at distance A. Accordingly, incorporating a landing zone 324 having an increased density of discrete islands 322 of adhesive may create a region of base membrane 310 that is able to more easily adhere to a tissue to which the adhesive covering will be applied.

As an example, distance A may be in the range of approximately 1 mm to approximately 2 mm, while the distance between rows of discrete islands 322 of adhesive in landing zone 324 may be in the range of approximately 0.5 to approximately 1 mm. For example, distance A may be approximately 1 mm, approximately 1.1 mm, approximately 1.2 mm, approximately 1.3 mm, approximately 1.4 mm, approximately 1.5 mm, approximately 1.6 mm, approximately 1.7 mm, approximately 1.8 mm, approximately 1.9 mm, or approximately 2 mm, The distance between rows in landing zone 324 may be in the range of approximately 0.5 mm, approximately 0.6 mm, approximately 0.7 mm, approximately 0.8 mm, approximately 0.9 mm, or approximately 1 mm.

As shown in FIG. 6, columns of discrete islands 322 of adhesive may be spaced apart from one another by a distance B. In one example, distance B between columns of discrete islands 322 of adhesive may be approximately 0.5 mm to approximately 1 mm, e.g., approximately 0.5 mm, approximately 0.6 mm, approximately 0.7 mm, approximately 0.8 mm, approximately 0.9 mm, or approximately 1 mm. Distance B may be determined, at least in part, based on the factors discussed above in relation to distance A, such as retention strength, the flow of nutrients and waste, ease of handling, etc.

In some aspects, a central region of base membrane 310 may have an increased spacing between columns of discrete islands 322. In FIG. 6, a distance D may separate two sections of rows of discrete islands 322 of adhesive. Distance D of base membrane 310 that is free of adhesive may be configured to overlay a portion of injured tissue, e.g., a coaptation zone in the case of transected tissue. In this manner, adhesive may not interfere with the healing of the injured or transected tissue or may not irritate the site of injury. Distance D may be approximately 1 mm to approximately 5 mm, depending on the size or diameter of the tissue to which it will be applied. As an example, for an adhesive covering designed for use with nerves, distance D may be approximately 1.5 mm for nerves with a diameter of approximately 1-3 mm, approximately 2.5 mm for nerves with a diameter of approximately 3-5 mm, and approximately 3.5 mm for nerves with a diameter of approximately 5 mm or larger, although other ranges are contemplated.

In the example of FIG. 6, discrete islands 322 of adhesive may not extend to the extreme edge of base membrane 310. In this embodiment, adhesive may be set back from the extreme edge a distance C. Distance C may be approximately 0.1 mm to approximately 1 mm, e.g., approximately 0.1 mm to approximately 0.5 mm, approximately 0.5 mm to approximately 1 mm, approximately 0.2 mm to approximately 0.8 mm, approximately 0.1 mm to approximately 0.2 mm, approximately 0.2 mm to approximately 0.3 mm, approximately 0.3 mm to approximately 0.4 mm, approximately 0.4 mm to approximately 0.5 mm, approximately 0.5 mm to approximately 0.6 mm, approximately 0.6 mm to approximately 0.7 mm, approximately 0.7 mm to approximately 0.8 mm, approximately 0.8 mm to approximately 0.9 mm, or approximately 0.9 mm to approximately 1 mm. A smaller border of base membrane 310 (e.g., a smaller distance C) may allow for more adhesive coverage and increased retention strength, e.g., coaptation strength, while a larger border (e.g., a larger distance C) may facilitate handling by a user. In some aspects, the selected border width may facilitate cutting of individual adhesive coverings to final size during manufacture without disturbing the pattern of discrete islands 322 of adhesive.

FIG. 6 also depicts a height F and a width E or individual discrete islands 322 of adhesive. In this example, height F and width E may be approximately 0.5 mm to approximately 2 mm, e.g., approximately 0.5 mm, approximately 1 mm, approximately 2 mm, approximately 0.75 to approximately 1.75, approximately 0.5 mm to approximately 1 mm, or approximately 1 mm to approximately 2 mm. It is theorized that the physiological diffusion limit may be on the order of approximately 0.5 mm for the adhesive material, which may facilitate nutrient and waste regulation, as described above, throughout a rectangle with a height and width of approximately 1 mm.

In some aspects, individual discrete islands 322 of adhesive may have a thickness of approximately 50 μm to approximately 500 μm, e.g., approximately 50 μm to approximately 250 μm, approximately 250 to approximately 500 μm, approximately 100 μm to approximately 400 μm, approximately 200 μm to approximately 300 μm, etc. In some aspects, maximum particle size of the adhesive may be on the order of approximately 50 μm, which may limit the minimum achievable thickness. Increasing the thickness too much may increase the shear that the adhesive experiences between the tissue surfaces and may lead to decreased retention strength, e.g., coaptation strength.

It will be understood that although examples of spacing distances and sizes of discrete islands 322 are described in reference to FIG. 6, such measurements are not limiting. For example, the measurements discussed in reference to FIG. 6 may be suitable for use with smaller tissues, such as nerves, and the spacing and sizes appropriate for a given adhesive covering of the disclosure may depend, at least in part, on the size or type of tissue to which an adhesive covering is intended for use with. Accordingly, the measurements are not limiting and are provided only as an example use case.

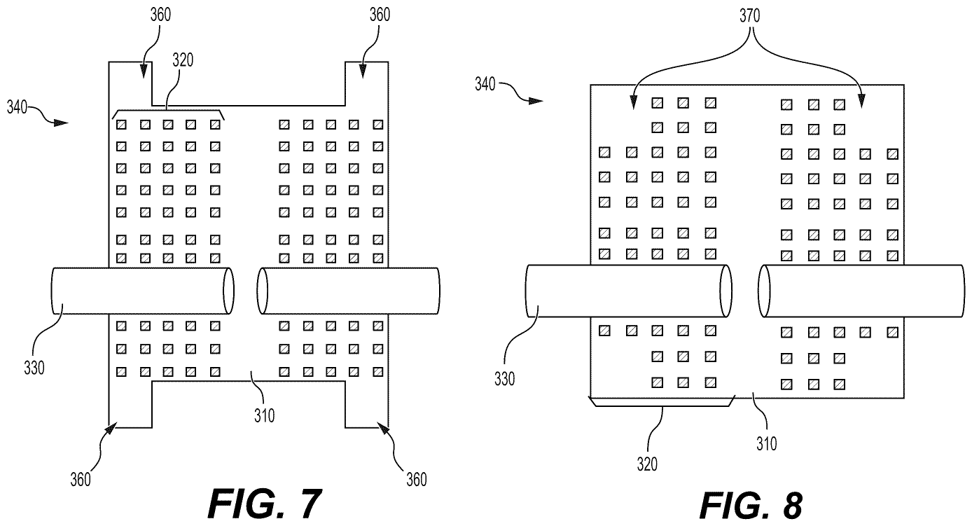

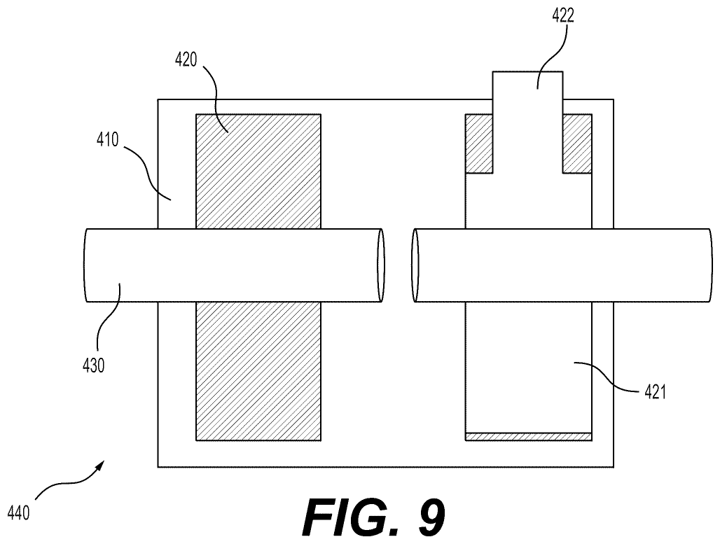

FIGS. 7 and 8 depict further exemplary arrangements of adhesive 320 on base membrane 310 and of shapes of base membrane 310. FIG. 7 depicts an adhesive covering 340 having tabs 360. Tabs 360 may be protruding portions of base membrane 310 or may be separate from base membrane 310. Accordingly, tabs 360 may be formed monolithically as part of base membrane 310, may be separate from base membrane 310 but formed of the same material as base membrane 310, or may be formed from a different material than base membrane 310. One or more tabs 360 may be free of adhesive and may facilitate manipulation of adhesive covering 340 by a user, e.g., with their fingers, forceps, or other surgical instruments, e.g., when applying adhesive covering 340 to underlying tissue. Although four tabs 360 are depicted in FIG. 7, more or fewer tabs 360 may be included as part of adhesive covering 340. Further, one or more tabs 360 may extend out from adhesive covering 340 in any suitable direction, e.g., in a direction perpendicular to tabs 360 depicted in FIG. 7, at an angle that is different from a longitudinal or latitudinal axis of adhesive covering 340, or a combination thereof. In some aspects, one or more tabs 360 may extend from one or more edges of adhesive covering 340 other than a corner portion, or in addition to a corner portion. Tabs 360 may be any suitable size or shape, e.g., elongated strips, rounded protrusions, bulbous protrusions, triangular protrusions, or combinations thereof. In some aspects, the adhesive coverings depicted in FIGS. 1A, 1B, 2A, 2B, 3A, 3B, 3C, 6, 8, or 9 may include one or more tabs.

FIG. 8 depicts an adhesive covering 340 having a plurality of adhesive-free gaps 370. Adhesive-free gaps 370 may be portions of base membrane 310 that are free of adhesive and may facilitate manipulation of adhesive covering 340 by a user, e.g., with their fingers, forceps, or other surgical instruments, e.g., when applying adhesive covering 340 to underlying tissue. Although four adhesive-free gaps 370 are depicted in FIG. 8, more or fewer adhesive-free gaps 370 may be included on base membrane 310. Further, one or more adhesive-free gaps 370 may be included along one or more edges of adhesive covering 340 other than a corner portion, or in addition to a corner portion. Adhesive-free gaps 370 may be any suitable size or shape, e.g., rectangular, rounded, triangular, irregular, or combinations thereof. In some aspects, the adhesive coverings depicted in FIGS. 1A, 1B, 2A, 2B, 3A, 3B, 3C, 6, 7, or 9 may include one or more adhesive-free gaps. In embodiments in which adhesive is arranged on the base membrane in a plurality of discrete islands, adhesive-free gaps may be formed by relatively greater spacing between adjacent discrete islands, or by the omission of discrete islands on a portion of base membrane. In embodiments in which adhesive is applied in a continuous manner, e.g., as shown in FIGS. 1A and 1B, adhesive-free gaps may be formed where the adhesive ends, or may be formed as indents, peninsulas, or islands of adhesive-free regions extending into adhesive regions.

In some of the embodiments described herein, adhesive may cover approximately 20% to approximately 75% of a given base membrane, e.g., approximately 30% to approximately 50%, approximately 20% to approximately 50%, or approximately 50% to approximately 75%. In some embodiments, however, adhesive may cover less than approximately 20% or more than approximately 75% of the base membrane. Adhesive coverage percentage may depend, at least in part, on the spacing of adhesive on the base membrane, such as the spacing of discrete islands 322 of adhesive on base membrane 310 (e.g., distances A, B, C, D). Higher coverage percentages may improve retention strength, e.g., coaptation strength, but may allow for smaller or fewer regions without adhesive. More adhesive coverage may in some aspects result in smaller zones without adhesive configured to overlay injured tissue or coaptation zones, or may leave less space for tissue swelling, less space for the flow of nutrients and waste, or may increase the difficulty of handling by a user.

As described above, adhesive 320 of FIGS. 3A-3C and FIGS. 6-8, and discrete islands 322 of adhesive of FIGS. 5A-5C, may be applied to base membrane 110 using one or more various methods, such as painting, printing (e.g., 3D printing, inkjet printing, screen printing), rolling, spraying, solvent casting, or any other methods of applying the adhesive. In some aspects, the adhesive may be applied to a base membrane using stencils to form reproducible patterns of adhesive on the base membrane. Such application methods may promote consistent deposition of adhesive material and scalable manufacturing.

In some examples, all or part of the adhesive may be covered with a liner to allow the user, e.g., a surgeon, to position the adhesive covering without the adhesive immediately sticking to the first thing it touched. For example, in the case of nerve coaptation, the liner may allow a surgeon to align the nerve ends, or other tissue, relative to the adhesive covering and relative to one another before removing the liner to expose the adhesive and allow the tissue to stick to the adhesive. If a tubular shape adhesive covering is used, the liner may allow a surgeon to insert tissue, e.g., nerve tissue, into opposing ends of the adhesive covering, without the adhesive being so sticky so as to make insertion of the tissue difficult or impossible. In some aspects, the liner may protect the adhesive from being accidentally activated by exposure to moisture in the surgical field before the user is ready to place the adhesive covering.

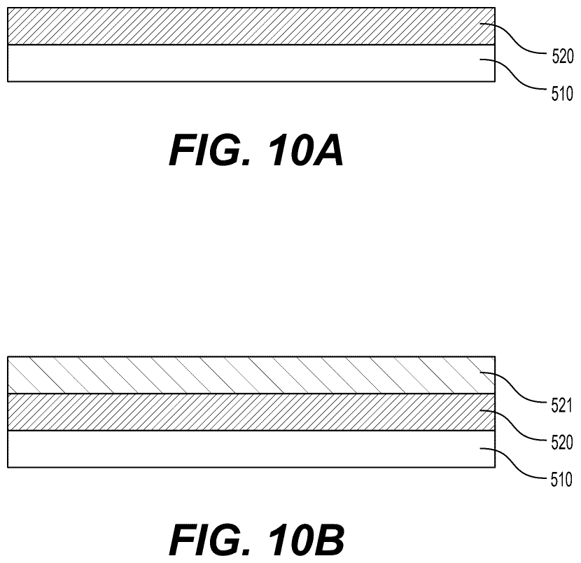

FIG. 9 depicts a liner 421 over one of two adhesive areas 420 of an adhesive covering in the form of a wrap. In some examples, the liner may include a tab 422 that may be grasped by the user to remove the liner 421 and expose the adhesive 420, once the adhesive nerve wrap has been properly placed relative to the nerve ends 430. In some examples, the liner may be comprised of flexible, biocompatible materials that do not interact with the underlying adhesive 420. Examples include natural or synthetic materials, such as waxed paper, polymers, etc.

In some aspects, rather than using a removable liner, or in addition to using a removable liner, an adhesive covering of the current disclosure may include a time control layer overlaying all or part of the adhesive. The time control layer may function in a similar manner as a removable liner, in that it allows the user, e.g., a surgeon, to position the adhesive nerve covering without the adhesive immediately sticking to the first thing it touched. For example, in the case of nerve coaptation, the liner may allow a surgeon to align the nerve ends relative to the adhesive covering and relative to one another before the adhesive is ready to stick to the adhesive. If a tubular shape adhesive covering is used, the time control layer may allow a surgeon to insert tissue, e.g., nerve tissue, into opposing ends of the adhesive covering, without the adhesive being so sticky right away so as to make insertion of the tissue difficult or impossible. However, as opposed to a removable liner, which needs to be removed after the adhesive covering is oriented relative to the tissue, the time control layer may dissolve to expose the adhesive without having to remove a liner.

In some aspects, the time control layer may be tuned to dissolve after approximately 5 seconds, approximately 10 seconds, approximately 15 seconds, approximately 20 seconds, approximately 30 seconds, approximately 45 seconds, approximately 60 seconds, approximately 90 seconds, or approximately 180 seconds. The time control layer may be configured to dissolve in about 5 seconds to about 30 seconds, about 5 seconds to about 20 seconds, about 5 seconds to about 15 seconds, about 15 seconds to about 60 seconds, about 30 seconds to about 60 seconds, about 30 seconds to about 90 seconds, about 30 seconds to about 180 seconds, about 15 seconds to about 90 seconds, about 15 seconds to about a minute, about 15 seconds to about 45 seconds, about 15 seconds to about 3 minutes, less than about 5 minutes, less than about 4 minutes, less than about 3 minutes, less than about 2 minutes, less than about 1 minute, or less than about 30 seconds.

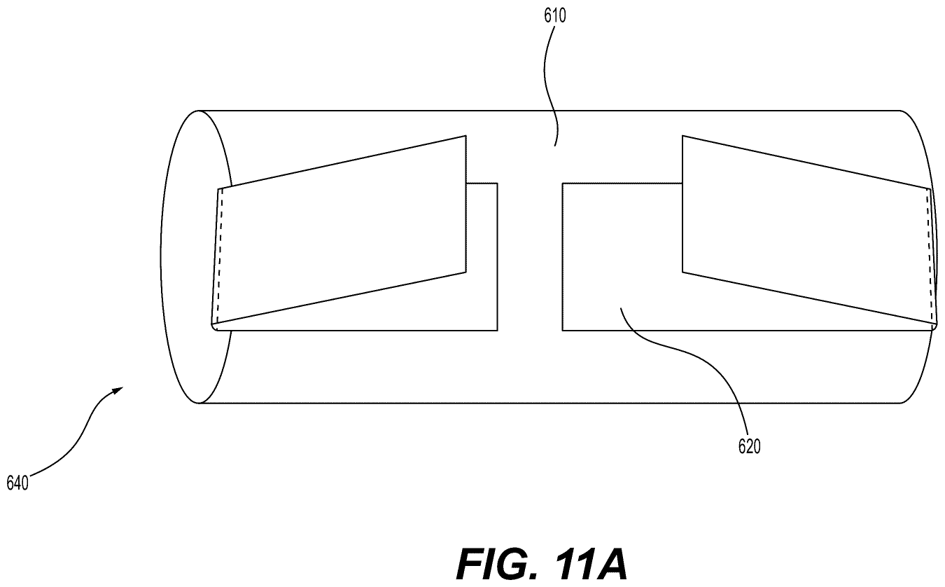

In some examples, the time control layer may be coated on top of or incorporated into the adhesive layer. In some examples, the time control layer may be applied by spraying a solution onto the adhesive surface or adding a separate film over it. According to aspects of the present disclosure, the time control layer may be applied to a coated surface designed to maintain the integrity of the adhesive through the application process. FIG. 10A depicts a schematic side view of a base membrane 510 with an adhesive 520 layered thereon, and FIG. 10B depicts a schematic side view of an additional time control layer 521 coated on top of the adhesive layer 520.

According to aspects of the disclosure, the time control layer may assist in regulating the binding of the adhesive to underlying tissue. In one example, the time control layer may assist in regulating the binding of an N-hydroxysuccinimide (NHS)-ester-based adhesive to the epineural proteins, in the case of application to a nerve. In some examples, the bonding process of the adhesive may depend on the interaction and reaction between the NHS-ester and free amine groups, forming a new amine bond. Once this bond is formed, repositioning the adhesive covering may cause damage to the nerve ends, specifically the delicate epineurium of the nerve, resulting in more damaged tissue, loss of previously healthy tissue, or less effective coaptation or healing. To delay this reaction, a sacrificial time control layer may be used to temporarily separate the reactants. This layer may be configured to dissolve and disperse after a short period of time, as described above, thus controlling the timing of the reaction. Accordingly, this time control layer may determine how long the adhesive layer may maintain working flexibility, such that the user may adjust the position of the adhesive covering relative to the tissue without causing damage to the tissue, e.g., nerve. By allowing the material of the time control layer to diffuse away from the adhesive-tissue interface, the reaction time may be extended, permitting longer manipulation of the adhesive nerve wrap and the tissue.

Suitable materials for use as the time control layer may include one or more of the following characteristics: high biocompatibility, high water solubility, fast absorbing, low molecular weight for rapid diffusion, capability to form a film on top of the adhesive layer, solubility in methanol, ethanol, acetone, or another volatile organic solvent, non-interference with NHS-ester chemistry, avoiding ester degradation and other characteristics that may assist in blocking the reaction between the adhesive and the tissue, e.g., nerve.

The time control layer may be formed, for example, out of natural or synthetic materials with a known and a tunable rate of dissolving when exposed to specific solvents. Examples of suitable materials include one or more of sugar alcohols, such as sorbitol/mannitol/xylitol/erythritol; PVA (polyvinyl alcohol); PVP (polyvinylpyrrolidone); PEG (Polyethylene glycol); PPG (polypropylene glycol); alginate; chondroitin sulfate; heparin; etc. In some examples, the time control layer may comprise a carboxylic acid and an amine group.

The liners described herein in regard to FIGS. 9 and 10B may be used in conjunction with any of the adhesive coverings described herein.

As described above, the adhesive covering may have a tubular shape, such as in the case of a nerve connector. Although a tubular shape is described, the tissue connectors may have a cross-sectional shape that is suitable for use with the tissue on which they are intended to be applied. For example, the adhesive covering may have an oval, a rectangular, a triangular, or any other suitable cross-sectional shape suitable to cover the target tissue or nerve.

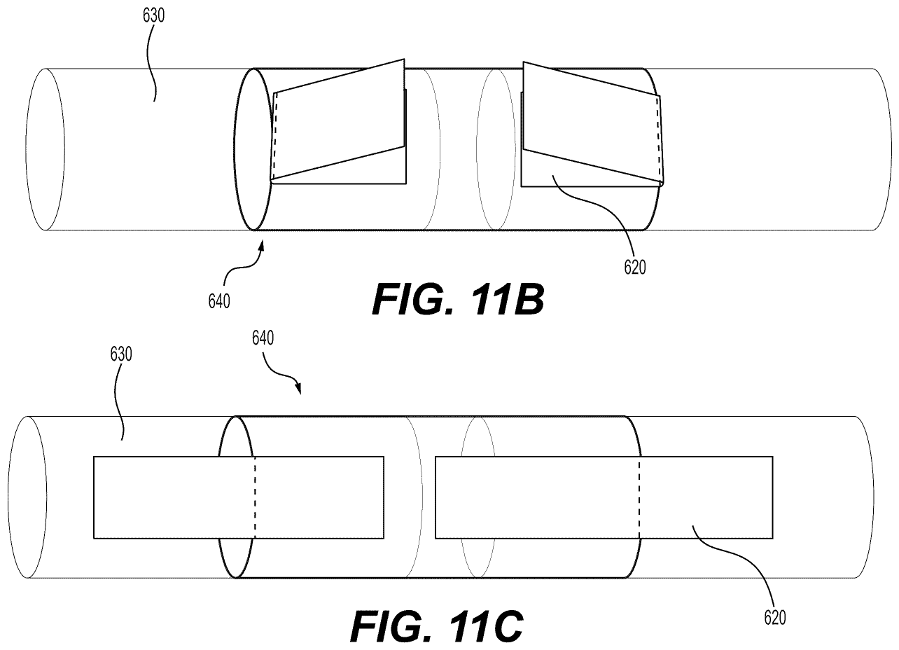

The tubular adhesive covering may include one or more adhesive strips incorporated onto, into, or otherwise with, the tubular adhesive covering, or monolithically formed as part of the base membrane of the tubular adhesive covering, in order to secure the tubular adhesive covering in place once tissue, e.g., nerves, have been inserted into the tubular adhesive covering. Such a tubular adhesive covering is depicted in FIG. 11A. If the adhesive strips are formed as part of the tubular adhesive covering, then the tubular portion of the tubular adhesive covering may include one or more flaps extending out from one or both ends of the tubular adhesive covering. A side of the flaps designed to face the tissue once the tissue is at least partially received within the tubular adhesive covering may include adhesive thereon. As described above, adhesive may or may not be covered by a liner or a time control layer. In some aspects, as shown in FIG. 11A, the adhesive strips 620 (in this case, formed as flaps of base membrane 610 extending out from ends of the tubular adhesive covering), may be designed to be biased away from openings of the tubular adhesive covering prior to use, so as to not interfere with the insertion of tissue into the tubular adhesive covering. Accordingly, they are shown folded back in FIG. 11A. Once tissue, e.g., nerve, is inserted into the tubular adhesive covering (FIG. 11B), the flaps may be extended generally along an axis of tubular adhesive covering 640 so as to contact the tissue proximal to a portion of the tissue that is received within tubular adhesive covering 640. If a liner is used over the flaps, the liner may be removed prior to contacting the flaps with the tissue. Once in place, the adhesive on the flaps may secure to the tissue to hold the tissue within the tubular adhesive covering 640.

In some aspects, using a plurality of spaced apart adhesive strips may allow for swelling or shrinking of the underlying tissue, as is described with reference to FIGS. 2A and 2B. The adhesive strips may space apart the adhesive, providing space in between adjacent adhesive strips for the tissue to expand and contract in a similar manner.

Although FIGS. 11A through 11C depict a tubular adhesive covering, adhesive strips may alternatively be incorporated as part of or monolithically formed as part of the base membrane of a non-tubular adhesive covering, such as a flat or pre-rolled sheet or wrap.

In other aspects, the adhesive strips 620 may be formed as separate structures from tubular adhesive covering 640 that are at least partially affixed to the tubular adhesive covering. For example, as shown in FIG. 11A, a portion of the adhesive strips 620 may be attached to an inner or outer surface of tubular adhesive covering 640, and a portion of the adhesive strips 620 may extend out from ends of tubular adhesive covering 640. Adhesive strips 620 may be formed of the same material as base membrane 610 of the tubular adhesive covering or of a different material. FIG. 11A depicts a tubular adhesive covering 640 with two adhesive strips 620 affixed to the inner portion of the nerve connector 610 and extending from ends of tubular adhesive covering 640, although any suitable number of adhesive strips 620 may extend from the ends of tubular adhesive covering 640. Further, although FIGS. 11A-11C describe and depict a tubular adhesive covering 640 used with adhesive strips 620, adhesive strips 620 may be integrated with or used with a wrap-like adhesive covering as described in FIGS. 1A-4 above, or any other non-tubular adhesive covering, such as a flat or pre-rolled sheet or wrap.

As described above, the adhesive strips 620 may be folded away or otherwise biased away from the openings of the tubular adhesive covering 640 to facilitate tissue insertion. FIG. 11B depicts nerve ends 630 inserted into the tubular adhesive covering 640 shown in FIG. 11A. The nerve ends 630 may be inserted into the openings of the tubular adhesive covering 640. The free ends of the adhesive strips 620 may then be unfolded and attached to the nerves 630 to secure the nerves 630 in place, and to secure the tubular adhesive covering 640 to the nerves 630 once the user is satisfied with the positioning of the nerve ends and the tubular adhesive covering 640.

FIG. 11C depicts the tubular adhesive covering 640 described in FIGS. 11A and 11B with the adhesive strips 620 unfolded to attach to the nerve ends 630. Though two adhesive strips are depicted, the present disclosure should not be limited to two adhesive strips. In some examples, the tubular adhesive covering 640 may include one or more adhesive strips, two or more adhesive strips, three or more adhesive strips, four or more adhesive strips, five or more adhesive strips, six or more adhesive strips, etc. In some examples, the number of adhesive strips may be determined at least in part by the size of the adhesive covering 640, the surface area of tissue to be contacted by the adhesive covering 640, the size, type, or location of tissue within the body it is to be applied to, etc.

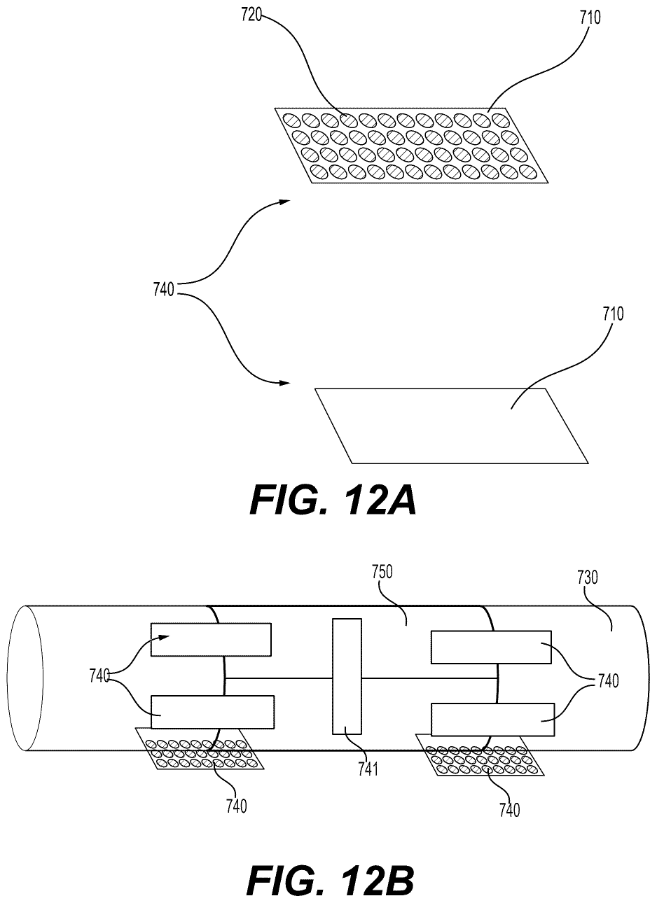

In some aspects, the adhesive strips described in reference to FIGS. 11A-11C may be individual strips that are separate from an adhesive covering and may be used in conjunction with either an adhesive covering or a traditional covering (e.g., a connector, a wrap, a protector, etc.,) that does not include a biocompatible and biodegradable adhesive. An exemplary adhesive strip 740 is shown in FIG. 12A. A first side of adhesive strip 740 may have adhesive 720 applied to it, and a second side of adhesive strip 740 may have no adhesive applied to it. The adhesive strip may be formed of a backing layer 710 and adhesive 720 applied to the backing layer 710. The adhesive may be applied in any of the manners described above. The backing layer 710 may be formed of any of the materials described above in reference to the base membranes of the prior embodiments, and the adhesive 720 may also be formed of any of the biocompatible adhesive materials described above.

As described above, the adhesive 720 of the adhesive strip 740 may have a clear color, a color that is similar to the backing layer 710, or a color that is different from the backing layer 710. In some aspects, the adhesive 720 may have a color that is different from the backing layer 710 so as to allow the user to see where the adhesive 720 is on the adhesive strip 740, and where to apply the adhesive strip 740 to the tissue. The different color may enhance visibility and help to determine the location of the adhesive 720 prior to application of the adhesive strip 740. The difference in color may also allow a user to identify which side of the adhesive strip 740 is the sticky side. In some aspects, the color may change to indicate to a user that the adhesive 720 has set, and thus the user should no longer try to reposition the adhesive strip 740. For example, in the case of nerves, the change in color once the adhesive 720 has set may prevent a user from trying to reposition the adhesive strip 740, which may otherwise damage the delicate epineurium layer.

The adhesive strips 740 described herein may be are engineered to bond with both natural and synthetic materials that serve as the form factor for tissue repair, e.g. wraps, connectors, protectors, tissues such as nerve, etc. Although shown in FIG. 12A as rectangular, the adhesive strips 740 may be formed in a range of shapes and sizes to conform to the specific requirements of the nerve or tissue to which it will be applied, or to the device it is configured for use with. For example, the adhesive strips may be rectangular, circular, oval, triangular, irregularly shaped, or other suitable shapes. In some aspects, the adhesive strip 740 may come as a continuous roll and may be cut according to the size needed.

The function of the adhesive strip 740 may be to secure a wrap, protector, or connector in place relative to tissue (e.g., nerve), to promote stable positioning and alignment. Alternatively, in some aspects, the adhesive strip may be used by itself, without a connector, protector, or wrap, e.g., to cover an injured portion of tissue on its own or to hold portions of tissue together. If used with a traditional connector, protector, or wrap, then use of the adhesive strips 740 may allow a traditional covering to achieve sutureless attachment, and may render sutures unnecessary, or fewer sutures may be needed. The dimensions and materials of the adhesive strip 740 may be designed to provide robust strength during repair or coaptation, e.g., to have comparable holding strength to sutures. The customization ensures that the adhesive strip devices are effective in securing a covering in place while being gentle enough to inhibit risk of damage to the nerve or tissue itself.

The adhesive strip 740 may be biocompatible and resorbable and able to adhere to tissue, to adhere to a device it is intended to be used with, or to itself. The backing layer 710 may be engineered to provide support to the adhesive 720 to inhibit deformation during application and to inhibit accidental adhesion to undesired surrounding surfaces. As such, the backing layer 710 may have some degree of rigidity. The rigidity of the backing layer 710 may promote precise placement and effectiveness in facilitating tissue, e.g., nerve repair. In some examples, the backing layer 710 may be comprised of ECM material, such as SIS, or a resorbable polymer membrane, although any material described as being suitable for the base membranes described herein may be used.

In some examples, to form the adhesive strip 740, the adhesive 720 may be coated or layered on one side of the backing layer 710, forming an adhesive layer. As described above, the adhesive layer may be used to attach the adhesive strip 740 securely to the target tissue without the use of traditional sutures, or with fewer sutures. The single-sided nature of the adhesive strip 740 may inhibit unnecessary sticking to adjacent tissues or structures. The adhesive 720 may be applied in any manner as described above and may be arranged on the adhesive strip 740 as described above, e.g., as a continuous application or in a pattern or as discrete islands of adhesive material. For example, the adhesive 720 may be applied as a linear strip, or as a series of linear strips. In other aspects, adhesive 720 may be applied in a series of discrete islands or as a regular or irregular pattern of discrete islands, e.g., as dots, drops, triangles, quadrilaterals, lines, star-with-dot motifs, zigzags, etc. In some aspects, adhesive 720 may be applied in serpentine patterns, as a continuous serpentine line or as a series of discrete lines, or as any other suitable shapes or arrangements. In some aspects, the size, shape, arrangement, or distribution of adhesive 720 may be determined, at least in part, on the type of tissue to which the adhesive strip 740 is intended to be applied, or the particular use case for the adhesive strip 740. In some examples, the adhesive 720 of the adhesive strip 740 may be coated in a time control layer or a liner to inhibit immediate attachment to the target tissue, as described above.

In some examples, on the side of the adhesive strip 740 without the adhesive 720, a lubricating layer may be applied to inhibit unwanted sticking of the adhesive strip 740 to surrounding surfaces near the site of tissue repair, e.g., nerve coaptation. In some examples, the lubricating layer may include hyaluronate or alginate. Suitable lubricating layers may be described in U.S. Patent Application Publication No. 2021/0046221, filed Aug. 13, 2020, which is incorporated by reference in its entirety herein.

FIG. 12B shows an example of adhesive strips 740 in use. FIG. 12B depicts adhesive strips 740 being used in conjunction with a nerve wrap 750. Specifically, the wrap has been placed around nerve 730, and adhesive strips 740 are placed at the ends of the wrap 750 to affix the ends of the wrap 750 to the nerve 730. The adhesive layer of the adhesive strips 740, illustrated with discrete islands on one side of the device, is placed on the outside of the ends of nerve wrap 750 and the nerve ends 730 to hold the two together. Further depicted in FIG. 12B, another adhesive strip 741 (which may functionally be the same as nerve wraps 740, but is indicated using a different reference number in order to identify its location) is applied to the end of the nerve wrap 750 to secure closure of the wrap 750. Adhesive strips 740 may accordingly be used in place of sutures for nerve repair or tissue repair more generally.

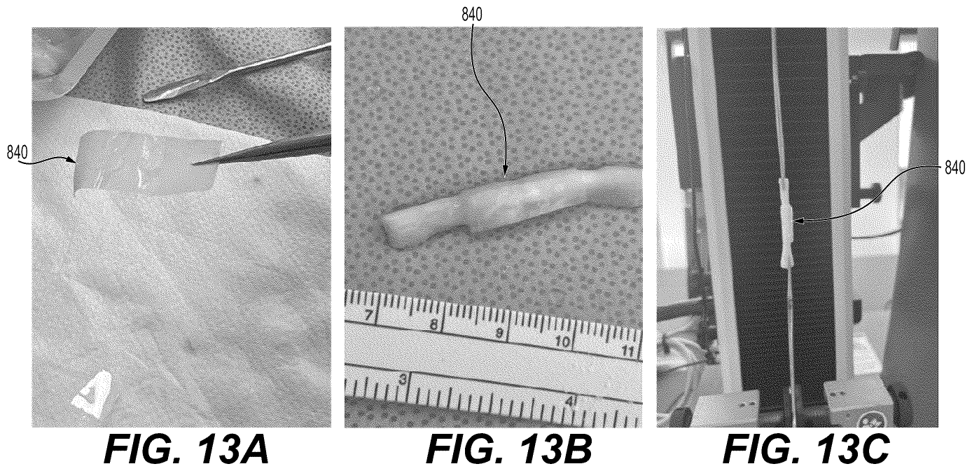

FIGS. 13A-13C illustrate an experimental use of a prototype adhesive covering in the form of an adhesive nerve wrap 840. FIG. 13A shows an example adhesive nerve wrap 840 with adhesive sites on the proximal and distal edges of the wrap. FIG. 13B shows the adhesive nerve wrap 840 applied to a transected nerve for a sutureless nerve repair. According to experimental results of the strength testing depicted in FIG. 13C, the adhesive nerve wrap 840 formed a sutureless nerve repair with a strength at 2.54 N, which is similar to the strength of sutures.

Embodiments of the disclosure that include biocompatible and biodegradable adhesive may afford benefits over existing methods for tissue fixation, such as reducing or eliminating the need for sutures, allowing for sutureless tissue or nerve repair procedures, such as coaptation.

Although adhesive coverings and adhesive strips of the present disclosure are described mainly for use with nerves, examples of tissue with which adhesive coverings and adhesive strips described herein may be used include nerve tissue, such as peripheral nerve tissue or central nervous system tissue, as well as other types of tissue. Exemplary tissue types suitable for use with adhesive coverings and adhesive strips include, but are not limited to, epithelial tissue, connective tissue, muscular tissue, tendon tissue, ligament tissue, vascular tissue, intestinal tissue, dermal tissue, and cardiac tissue. The tissue may be mammalian tissue, including human tissue and tissue of other primates, rodent tissue, equine tissue, canine tissue, rabbit tissue, porcine tissue, or ovine tissue. In some aspects, the tissue may be non-mammalian tissue, selected from piscine, amphibian, or insect tissue. The tissue may be a synthetic tissue, such as, but not limited to, laboratory-grown or 3D-printed tissue.

It should be understood that although the present disclosure has been made with reference to preferred embodiments, exemplary embodiments, and optional features, modifications and variations of the concepts herein disclosed may be resorted to by those skilled in the art, and that such modifications and variations are considered to be within the scope of this disclosure as defined by the appended claims. The specific embodiments and examples provided herein are examples of useful embodiments of the present disclosure and are non-limiting and illustrative only. It will be apparent to one skilled in the art that the present disclosure may be carried out using a large number of variations of the devices, device components, methods, and steps set forth in the present description. As will be recognized by one of skill in the art, methods and devices useful for the present methods can include a large number of various optional compositions and processing elements and steps.

We claim:

1. An adhesive tissue covering comprising:
a base membrane; and
an adhesive, wherein the adhesive is biodegradable and biocompatible and is configured to bond to tissue, and wherein the adhesive is located along one or more edges of the base membrane.

2. The adhesive tissue covering of claim 1, further comprising a time control layer coated on the adhesive.

3. The adhesive tissue covering of claim 2, wherein the time control layer is comprised of at least one of a sugar alcohol, sorbitol, mannitol, xylitol, erythritol, PVA (polyvinyl alcohol), PVP (polyvinylpyrrolidone), PEG (Polyethylene glycol), PPG (polypropylene glycol), alginate, chondroitin sulfate, heparin, a carboxylic acid, or an amine group.

4. The adhesive tissue covering of claim 1, further comprising a non-reactive liner positioned over the adhesive.

5. The adhesive tissue covering of claim 1, wherein the adhesive is formed as a continuous strip on at least half of at least one edge of the base membrane.

6. The adhesive tissue covering of claim 1, wherein the adhesive is formed as a plurality of discrete islands along at least one edge of the base membrane.

7. The adhesive tissue covering of claim 6, wherein the adhesive extends along less than half of at least once edge of the base membrane.

8. The adhesive tissue covering of claim 6, wherein the base membrane includes longitudinal slits along at least one edge of the base membrane.

9. The adhesive tissue covering of claim 1, wherein the adhesive is applied to the base membrane by painting, printing, rolling, spraying, or solvent casting.

10. The adhesive tissue covering of claim 1, wherein the adhesive tissue covering is an adhesive nerve covering.

11. The adhesive tissue covering of claim 1, wherein the base membrane comprises a tissue-based material.

12. The adhesive tissue covering of claim 11, wherein the tissue-based material is an amnion-based tissue.

13. The adhesive tissue covering of claim 11, wherein the tissue-based material is small intestine submucosa.

14. The adhesive tissue covering of claim 1, wherein the base membrane comprises a resorbable polymer.

15. The adhesive tissue covering of claim 1, wherein the adhesive covers approximately 20% to approximately 75% of the base membrane.

16. An adhesive tissue strip comprising:
a base membrane formed of at least one of a tissue-based material or a resorbable polymer; and
an adhesive, wherein the adhesive is biodegradable and biocompatible and is configured to bond to tissue, and wherein the adhesive is located on one side of the base membrane.

17. The adhesive tissue strip of claim 16, further comprising a time control layer coated on the adhesive.

18. The adhesive tissue strip of claim 17, wherein the time control layer is comprised of at least one of a sugar alcohol, sorbitol, mannitol, xylitol, erythritol, PVA (polyvinyl alcohol), PVP (polyvinylpyrrolidone), PEG (Polyethylene glycol), PPG (polypropylene glycol), alginate, chondroitin sulfate, heparin, a carboxylic acid, or an amine group.

19. The adhesive tissue strip of claim 16, further comprising a non-reactive liner positioned over the adhesive.

20. The adhesive tissue strip of claim 16, wherein the adhesive is formed as a discrete pattern of geometric shapes.

21. The adhesive tissue strip of claim 16, wherein the adhesive is applied to the base membrane by painting, printing, rolling, spraying, or solvent casting.

22. The adhesive tissue strip of claim 16, wherein the adhesive tissue strip is an adhesive nerve strip.

23. The adhesive tissue strip of claim 16, wherein the base membrane comprises a tissue-based material, and wherein the tissue-based material is an amnion-based tissue.

24. The adhesive tissue strip of claim 16, wherein the base membrane comprises a tissue-based material, and wherein the tissue-based material is small intestine submucosa.
